(19) United States
(12) Patent Application Publication
(10) Pub. No.: US 2024/0379231 A1
(43) Pub. Date: Nov. 14, 2024

(54) DEEP NEURAL NETWORK ECG DATA ANALYSIS

(71) Applicant: Boston Scientific Cardiac Diagnostics, Inc., Rochester, MN (US)

(72) Inventors: Michael Thomas Edward McRoberts, Mazeppa, MN (US); David R. Engebretsen, Cannon Falls, MN (US)

(21) Appl. No.: 18/657,018

(22) Filed: May 7, 2024

Related U.S. Application Data
(60) Provisional application No. 63/464,703, filed on May 8, 2023.

(51) Int. Cl. G16H 50/20

(52) U.S. Cl. CPC G16H 50/20

(57) ABSTRACT

Systems and methods involve generating, by one or more machine learning models, a first set of classifications for a first strip of electrocardiogram (ECG) data; generating, by the one or more machine learning models, a second set of classifications for a second strip of ECG data; generating, by the one or more machine learning models, one or more new classifications in response to inputting only a portion of the first strip of ECG data along with only a portion of the second strip of ECG data into the machine learning model; and updating the first set of classification and/or the second set of classifications with the one or more new classifications.

### CROSS REFERENCE TO RELATED APPLICATION

This application claims priority to Provisional Application No. 63/464,703, filed May 8, 2023, which is herein incorporated by reference in its entirety.

### TECHNICAL FIELD

The present disclosure relates to devices, methods, and systems for monitoring, identifying, and classifying cardiac events.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Cardiac monitoring can be used, for example, to identify abnormal cardiac rhythms, so that critical alerts can be provided to patients, physicians, or other care providers and patients can be treated.

### SUMMARY

In Example 1, a method includes generating, by one or more machine learning models, a first set of classifications for a first strip of electrocardiogram (ECG) data; generating, by the one or more machine learning models, a second set of classifications for a second strip of ECG data; generating, by the one or more machine learning models, one or more new classifications in response to inputting only a portion of the first strip of ECG data along with only a portion of the second strip of ECG data into the machine learning model; and updating the first set of classification and/or the second set of classifications with the one or more new classifications.

In Example 2, the method of Example 1, wherein the portion of the first strip of ECG data and the portion of the second strip of ECG data comprise a beat that starts in the first strip and ends in the second strip.

In Example 3, the method of Example 2, wherein the one or more new classifications includes a classification of the beat that starts in the first strip and ends in the second strip.

In Example 4, the method of Examples 1-3, wherein the first set of classifications include a first set of beat classifications associated with beats within the first strip, wherein the second set of classifications include a second set of beat classifications associated with beats within the second strip, wherein the one or more new classifications are beat classifications for one or more beats that overlap the first strip and the second strip.

In Example 5, the method of any of Examples 1-4, further including: generating, by the one or more machine learning models, a first rhythm classification associated with the first strip of ECG data.

In Example 6, the method of Example 5, further including: updating the first rhythm classification with a new rhythm classification based on the one or more new classifications.

In Example 7, the method of Example 6, wherein the updating the first rhythm classification with the new rhythm classification is further based on at least some of the first set of classifications.

In Example 8, the method of any of Examples 1-7, wherein the first strip of ECG data comprises 1-10 minutes of ECG data, wherein the second strip of ECG data comprises 1-10 minutes of ECG data.

In Example 9, the method of any of Examples 1-8, wherein the portion of the first strip of ECG data contains 0.5-3 seconds of ECG data, wherein the portion of the second strip of ECG data contains 0.5-3 seconds of ECG data.

In Example 10, the method of any of Examples 1-9, wherein the portion of the first strip of ECG data and the portion of the second strip comprise a continuous section of the ECG data.

In Example 11, the method of any of Examples 1-10, wherein the one or more machine learning models comprise a deep convolutional neural network and/or a deep fully connected neural network.

In Example 12, the method of any of Examples 1-11, wherein the updating the first set of classification and/or the second set of classifications with the one or more new classifications comprises updating only a subset of the first set of classification and/or the second set of classifications.

In Example 13, a computer program product comprising instructions to cause one or more processors to carry out the steps of the method of Examples 1-12.

In Example 14, a computer-readable medium having stored thereon the computer program product of Example 13.

In Example 15, a computer comprising the computer-readable medium of Example 14.

In Example 16, a system includes a server comprising one or more processors and computer-readable media having computer-executable instructions. The computer-executable instructions are configured to be executed by the one or more processors to cause the server to: generate, by one or more machine learning models, a first set of classifications for a first strip of ECG data; generate, by the one or more machine learning models, a second set of classifications for a second strip of ECG data; generate, by the one or more machine learning models, one or more new classifications in response to inputting only a portion of the first strip of ECG data along with only a portion of the second strip of ECG data into the machine learning model; and update the first set of classification and/or the second set of classifications with the one or more new classifications.

In Example 17, the system of Example 16, wherein the portion of the first strip of ECG data and the portion of the second strip of ECG data comprise a beat that starts in the first strip and ends in the second strip.

In Example 18, the system of Example 17, wherein the one or more new classifications includes a classification of the beat that starts in the first strip and ends in the second strip.

In Example 19, the system of Example 16, wherein the first set of classifications include a first set of beat classifications associated with beats within the first strip, wherein the second set of classifications include a second set of beat classifications associated with beats within the second strip, wherein the one or more new classifications are beat classifications for one or more beats that overlap the first strip and the second strip.

In Example 20, the system of Example 16, wherein the computer-executable instructions are configured to be executed by the one or more processors to cause the server to: generate, by the one or more machine learning models, a first rhythm classification associated with the first strip of ECG data.

In Example 21, the system of Example 20, wherein the computer-executable instructions are configured to be executed by the one or more processors to cause the server to: update the first rhythm classification with a new rhythm classification based on the one or more new classifications.

In Example 22, the system of Example 21, wherein the updating the first rhythm classification with the new rhythm classification is further based on at least some of the first set of classifications.

In Example 23, the system of Example 16, wherein the portion of the first strip of ECG data contains 0.5-3 seconds of ECG data, wherein the portion of the second strip of ECG data contains 0.5-3 seconds of ECG data.

In Example 24, the system of Example 23, wherein the first strip of ECG data comprises 1-10 minutes of ECG data, wherein the second strip of ECG data comprises 1-10 minutes of ECG data.

In Example 25, the system of Example 16, wherein the one or more machine learning models comprise a deep convolutional neural network and/or a deep fully connected neural network.

In Example 26, the system of Example 25, wherein the update to the first set of classification and/or the second set of classifications with the one or more new classifications comprises updating only a subset of the first set of classification and/or the second set of classifications.

In Example 27, the system of Example 16, wherein the portion of the first strip of ECG data and the portion of the second strip comprise a continuous section of the ECG data.

In Example 28, a method includes generating, by one or more machine learning models, a first set of classifications for a first strip of ECG data; generating, by the one or more machine learning models, a second set of classifications for a second strip of ECG data; generating, by the one or more machine learning models, one or more new classifications in response to inputting only a portion of the first strip of ECG data along with only a portion of the second strip of ECG data into the machine learning model; and updating the first set of classification and/or the second set of classifications with the one or more new classifications.

In Example 29, the method of Example 28, wherein the portion of the first strip of ECG data and the portion of the second strip of ECG data comprise a beat that starts in the first strip and ends in the second strip, wherein the one or more new classifications includes a classification of the beat that starts in the first strip and ends in the second strip.

In Example 30, the method of Example 28, further including: generating, by the one or more machine learning models, a first rhythm classification associated with the first strip of ECG data; and updating the first rhythm classification with a new rhythm classification based on the one or more new classifications.

In Example 31, the method of Example 30, wherein the updating the first rhythm classification with the new rhythm classification is further based on at least some of the first set of classifications.

In Example 32, the method of Example 28, wherein the portion of the first strip of ECG data contains 0.5-3 seconds of ECG data, wherein the portion of the second strip of ECG data contains 0.5-3 seconds of ECG data.

In Example 33, the method of Example 28, wherein the portion of the first strip of ECG data and the portion of the second strip comprise a continuous section of the ECG data.

In Example 34, the method of Example 28, wherein the one or more machine learning models comprise a deep convolutional neural network and/or a deep fully connected neural network.

In Example 35, the method of Example 28, wherein the updating the first set of classification and/or the second set of classifications with the one or more new classifications comprises updating only a subset of the first set of classification and/or the second set of classifications.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, a remote computer, and a user interface, in accordance with certain instances of the present disclosure.

FIG. 3 shows examples of strips of ECG data, in accordance with certain instances of the present disclosure.

FIG. 4 shows a block diagram depicting an illustrative method, in accordance with certain instances of the disclosure.

FIG. 5 is a block diagram depicting an illustrative computing device, in accordance with instances of the disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Electrocardiogram (ECG) data of a patient can be used to analyze a patient's cardiac activity and recommend treatments. To collect ECG data, one or more monitoring devices (e.g., a sensor with electrodes) can be coupled to the patient such that the monitoring devices sense and record the ECG data. Using this approach, days of ECG data can be collected. However, days of ECG data can be overwhelming to store, process, and analyze locally by the monitoring devices or mobile devices. As such, the ECG data may be transmitted to a computing system such as a server that has more computational resources and storage resources.

The computing system may be programmed to input the ECG data into one or more machine learning models that are operated by the computing system and that are trained to analyze the ECG data. For time and/or computation efficiency, rather than process the ECG data as one large, continuous chunk of data; the one or more machine learning models may process the ECG data in smaller chunks of ECG data. For example, the entire patient study may comprise many separate individual strips of 1- to 10-minute intervals (or other time intervals) of ECG data, and the one or more machine learning models may process the individual strips separately.

However, splitting the ECG data into smaller individual strips potentially results in incomplete data at the boundaries of the strips. For example, an initial portion of a heartbeat (and/or rhythm) may start near the end of a first strip of ECG data, and a later portion of the heartbeat (and/or rhythm) may end near the beginning of a second, adjacent strip of ECG data. As such, the one or more machine learning models may process only a portion of a heartbeat (and/or rhythm) which may result in an inaccurate classification or the inability to even generate a classification for the partial heartbeat. Over the course of a patient study, these partial heartbeats (and/or rhythms) can create inaccurate or missed classifications. Instances of the present disclosure are accordingly directed to systems, methods, and devices for addressing inaccurate or missed classifications resulting from boundaries of strips of ECG data.

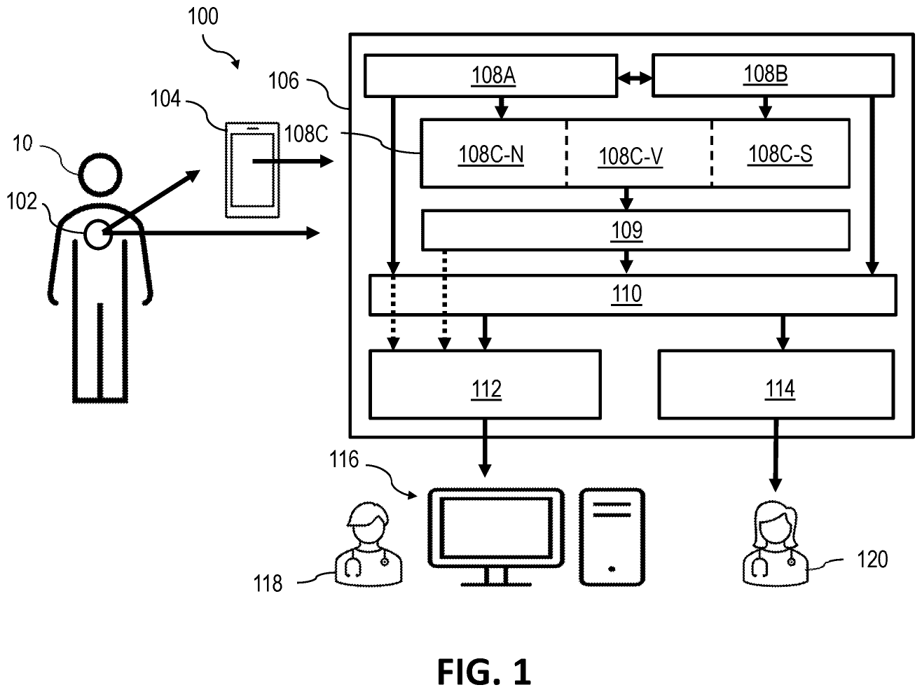

FIG. 1 illustrates a patient 10 and an example system 100. The system 100 includes a monitor 102 attached to the patient 10 or implanted in the patient 10 (e.g., pacemaker, ICD, CRT, or ICM) to detect cardiac activity of the patient 10. The monitor 102 may produce electric signals that represent the cardiac activity in the patient 10. For example, the monitor 102 may detect the patient's heart beating (e.g., using infrared sensors, electrodes, heart sounds) and convert the detected heartbeat into electric signals representing ECG data. In certain instances, the monitor 102 stores the ECG data of a patient study (e.g., one or more days of ECG data), after which the ECG data is transmitted to another device or system such as a server. Additionally or alternatively, the monitor 102 transmits the ECG data to a mobile device 104 (e.g., a mobile phone). In such instances, the mobile device 104 can include a program (e.g., mobile phone application) that receives, processes, and analyzes the ECG data. For example, the program may analyze the ECG data and detect or flag cardiac events (e.g., periods of irregular cardiac activity) contained within the ECG data.

The mobile device 104 can periodically transmit chunks of the ECG data to another device or system such as a server, which can process, append together, and store the chunks of the ECG data and metadata (e.g., time, duration, beat classifications, rhythm classifications for detected cardiac events) associated with the chunks of ECG data. In certain instances, the monitor 102 may be programmed to transmit the ECG data directly to the other device or system without utilizing the mobile device 104. Also, the monitor 102 and/or the mobile device 104 includes a button or touch-screen icon that allows the patient 10 to initiate an event. Such an indication can be recorded and communicated to the other device or system. In other instances involving multi-day studies, the ECG data and associated metadata are transmitted in larger chunks (e.g., an entire study's worth of ECG data).

### Cardiac Event Server

The ECG data (and associated metadata, if any) is transmitted to and stored by a cardiac event server 106 (hereinafter “the server 106” for brevity). The server 106 includes multiple models, platforms, layers, or modules that work together to process and analyze the ECG data such that cardiac events can be detected, filtered, prioritized, and ultimately reported to a patient's physician for analysis and treatment. In the example of FIG. 1, the server 106 includes one or more machine learning models 108A, 108B, and 108C, a clustering algorithm module 109, a cardiac event router 110, a report platform 112, and a notification platform 114. Although only one server 106 is shown in FIG. 1, the server 106 can include multiple separate physical servers, and the various models/platforms/modules/layers can be distributed among the multiple servers. Each of the models/platforms/modules/layers can represent separate programs, applications, and/or blocks of code where the output of one of the models/platforms/modules/layers is an input to another of the models/platforms/modules/layers. Each of the models/platforms/modules/layers can use application programming interfaces to communicate between or among the other models/platforms/modules/layers as well as systems and devices external to the server 106.

In certain instances, once the ECG data is processed by the machine learning models 108A-C and the clustering algorithm module 109, the ECG data (and associated metadata) is made available for the report platform 112. As will be described in more detail below, the report platform 112 can be accessed by a remote computer 116 (e.g., client device such as a laptop, mobile phone, desktop computer, and the like) by a user at a clinic or lab 118. In other instances, the cardiac event router 110 is used to determine what platform further processes the ECG data based on the classification associated with the cardiac event. For example, if the identified cardiac event is critical or severe, the cardiac event router 110 can flag or send the ECG data, etc., to the notification platform 114. The notification platform 114 can be programmed to send notifications (along with relevant ECG data and associated metadata) immediately to the patient's physician/care group remote computer 116 and/or to the patient 10 (e.g., to their computer system, e-mail, mobile phone application).

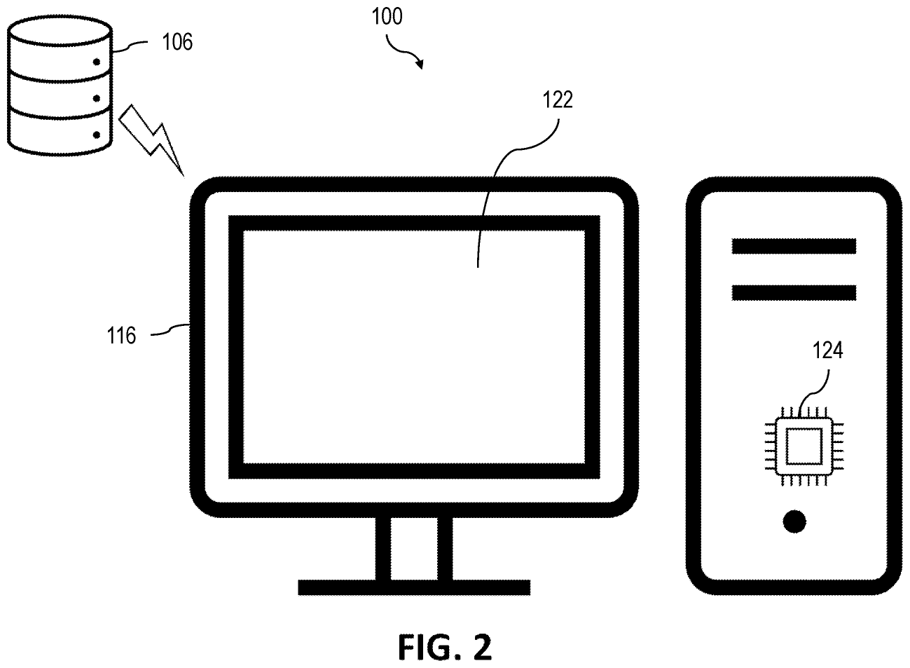

FIG. 2 shows the server 106 communicatively coupled (e.g., via a network) to the remote computer 116. In the example of FIG. 2, the remote computer 116 includes a monitor showing a user interface 122 that displays features of the report platform 112 hosted by the server 106. The user interface 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the user interface 122 to log into the report platform 112 via a web browser such that the user can use and interact with the report platform 112.

### Machine Learning Models

The server 106 applies the one or more machine learning models 108A-C to the ECG data to analyze and classify the beats and cardiac activity of the patient 10.

The first and second machine learning models 108A and 108B are programmed to—among other things—compare the ECG data to labeled ECG data to determine which labeled ECG data the ECG data most closely resembles. The labeled ECG data may identify a particular cardiac event and rhythm classification—including but not limited to ventricular tachycardia, bradycardia, atrial fibrillation, pause, normal sinus rhythm, or artifact/noise—as well as particular beat classifications—including but not limited to ventricular, normal, or supraventricular beats. In addition to identifying beat classifications and event classifications (and generating associated metadata), the first and second machine learning models 108A and 108B can determine and generate metadata regarding heart rates, duration, and beat counts of the patient 10 based on the ECG data. As specific examples, the first and/or the second machine learning models 108A and 108B can identify the beginning, center, and end of individual beats (e.g., individual T-waves) such that individual beats can be extracted from the ECG data. Each individual beat can be assigned a value (e.g., a unique identifier) such that individual beats can be identified and associated with metadata throughout processing and analyzing the ECG data.

The ECG data (e.g., ECG data associated with individual beats) as well as certain outputs of the first and second machine learning models 108A and 108B can be inputted to the third machine learning model 108C. Although two machine learning models are shown and described, a single machine learning model could be used to generate the metadata described herein, or additional machine learning models could be used.

The first and second machine learning models 108A and 108B can include the neural networks described in Ser. No. 16/695,534, which is hereby incorporated by reference in its entirety. The first neural network can be a deep convolutional neural network and the second neural network is a deep fully-connected neural network—although other types and combinations of machine learning models can be implemented. The first machine learning model 108A receives one or more sets of beats (e.g., beat trains with 3-10 beats) from individual strips of ECG data which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The second machine learning model 108B receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

The third machine learning model 108C (e.g., one or more trained encoder machine learning models) is programmed to generate latent space representations of the ECG data such that the ECG data is represented by fewer datapoints than the original ECG data. The latent space representations can be used as an approximation of the original raw ECG data for each beat.

In certain instances, instead of a single third machine learning model 108C, the server 106 includes a separate machine learning model for each type of beat classification (e.g., normal beats, ventricular beats, and supraventricular beats). For example, as shown in FIG. 1, the server 106 may include three third machine learning models (108C-N, 108C-V, and 108C-S) instead of a single third machine learning model. In certain instances, beats that were not initially classified (e.g., unclassified beats) can be processed either by a different third machine learning model or can skip the step of generating latent space representations and being clustered with similar shaped beats.

In the example of FIG. 1, one machine learning model 108C-N is used for beats classified as normal beats, another machine learning model 108C-V is used for beats classified as ventricular beats, and another machine learning model 108C-S is used for beats classified as supraventricular beats. As such, only ECG data (e.g., T-waves) of beats initially classified as normal beats by the first and/or second machine learning models 108A, 108B—as well as metadata generated by such machine learning models—are inputted to the machine learning model 108C-N, and so on.

Each third machine learning model (108C-N, 108C-V, 108C-S) receives ECG data associated with individual beats (e.g., an individual clip of ECG data for each beat) and generates latent space representations of such ECG data. For example, each individual beat is processed by one of the third machine learning models-depending on each individual beat's classification-such that the ECG data is distilled down to (or represented by) a small number of individual data points. Raw ECG data of an individual beat can include 500 or so datapoints, and each third machine learning model can distill the ECG data for a given beat into 4-16 datapoints.

The resulting datapoints are representations of an amplitude of the ECG signal at different relative points in time. These limited datapoints are datapoints that the trained machine learning models generate such that different beat shapes can be identified and similar shaped beats can be grouped together. Put another way, these datapoints may be those that are the most likely to be helpful in distinguishing among beat shapes.

The output(s) of the third machine learning model(s) 108C are processed by a clustering algorithm module 109. The clustering algorithm module 109 receives the latent space representations of individual beats and is programmed to associate similar shaped beats into different groups.

### Addressing Data Near Boundaries of ECG Strips

As previously noted, a patient study may comprise many separate individual strips of 1- to 10-minute intervals (or other time intervals) of ECG data, and the machine learning models may process the individual strips separately. However, splitting the ECG data into smaller individual strips can result in incomplete data at the boundaries of the strips.

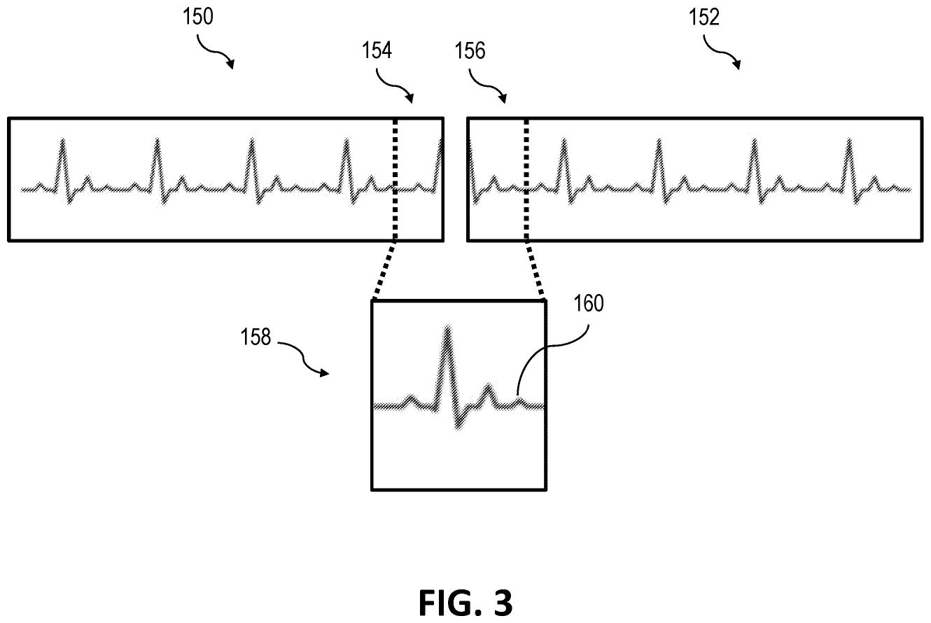

FIG. 3 shows an example of a situation where the ECG data at the boundaries of strips is incomplete. The left side of FIG. 3 shows a first strip 150 of ECG data, and the right side of FIG. 3 shows a separate, second strip 152 of ECG data. Near the end of the first strip 150, the plot of ECG data within the first strip 150 shows the beginning portion of a heartbeat. This heartbeat starts in the first strip 150 and ends in the second strip 152. Put another way, an end portion 154 of the first strip 150 of ECG data contains part of a heartbeat while an initial portion 156 of the second strip 152 contains the other part of the heartbeat. As such, the heartbeat spans over two separate strips of ECG data. In addition to the heartbeat spanning over two separate strips of ECG data, the patient may have experienced a cardiac event (e.g., an abnormal rhythm) that spans over two or more separate strips of ECG data. These conditions at the boundaries of the strips of ECG data can lead to inaccurate or missed classifications of beats and rhythms. For example, because beat classifications can affect the determine rhythm classification, an inaccurate beat classification can lead to an inaccurate rhythm classification.

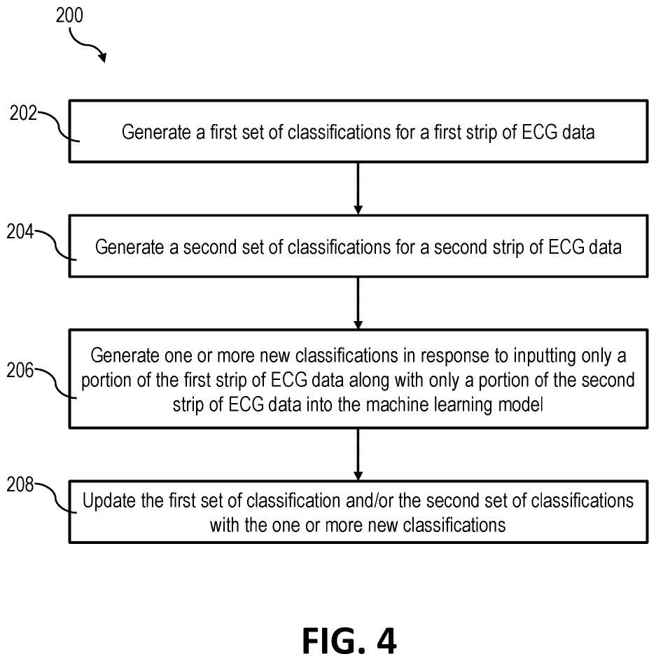

To help address such issues, a method 200 described herein and outlined in FIG. 4 can be utilized. The method 200 includes first inputting separate strips of ECG data into one or more machine learning models (such as those described herein) to generate respective classifications of the inputted ECG data. For example, the machine learning model(s) can generate a first set of classifications for the first strip of ECG data (block 202 in FIG. 4) and can generate a second set of classifications for the second strip of ECG data (block 204 in FIG. 4).

In certain instances, the classifications comprise classifications of individual beats within the ECG data. For example, each beat analyzed by the machine learning model(s) can be classified as a normal beat, a ventricular beat, a supraventricular beat, or an unclassified beat. Additionally or alternatively, the classifications comprise classifications of cardiac events such as type of rhythms (e.g., atrial fibrillation) that occurred within the strips of ECG data. The classifications can be generated for each strip of ECG data without reference to data from other strips of ECG data. Put another way, the classifications generated for the first strip of ECG data can be based solely on processing the first strip of ECG data by the one or more machine learning models.

After the first strip of ECG data and the second strip of ECG data have been separately processed by the one or more machine learning models, portions of the first strip and the second strips near the boundaries can be combined and processed again by the one or more machine learning models. Using FIG. 3 as an example, a new strip of ECG data 158 can be created by combining the end portion 154 of the first strip of ECG data 150 with the initial portion 156 of the second strip of ECG data 152. This new strip of ECG data 158 can contain a continuous section of ECG data for a full beat that otherwise was split into partial beats at the boundaries of the first strip 150 and the second strip 152.

The new strip of ECG data 158 can be inputted into the one or more machine learning models, which can generate one or more new classifications (e.g., beat classifications) (block 206 in FIG. 4). In certain instances, the new strip of ECG data 158 is processed using the same approach as that used when processing the first strip 150 and the second strip 152. In certain instances, the new strip 158 is a combination of 0.5-3 seconds of ECG data from the first strip 150 and 0.5-3 seconds of ECG data from the second strip 152. By combining a small fraction (e.g., 0.1-1%) of each of the larger strips of ECG data, the time and computational resources associated with re-running the machine learning models can be limited.

Once the one or more new classifications are generated, the new classifications can be used to replace one or more original classifications generated by the machine learning models for the first strip 150 and the second strip 152 (block 208 in FIG. 4). For example, if the partial beat was originally classified as a ventricular beat, that classification would be replaced by the new classification in the event the new classification was different than the original classification.

In addition to updating one or more of the original beat classifications with a new beat classification, rhythm classifications associated with the first strip 150 and the second strip 152 can be updated based on the new beat classification. As such, once the beat classifications have been updated, the process for assigning rhythm classifications to cardiac events can be run (or re-run) based on the new beat classifications.

Although the examples described above feature just two adjacent strips of ECG data, the approaches described herein can be applied to an entire study of ECG data. For example, with days of ECG split into smaller chunks, there will be thousands of boundaries of adjacent strips of ECG data that can be combined into much smaller strips and inputted into the one or more machine learning models to generate new classifications.

### Computing Devices and Systems

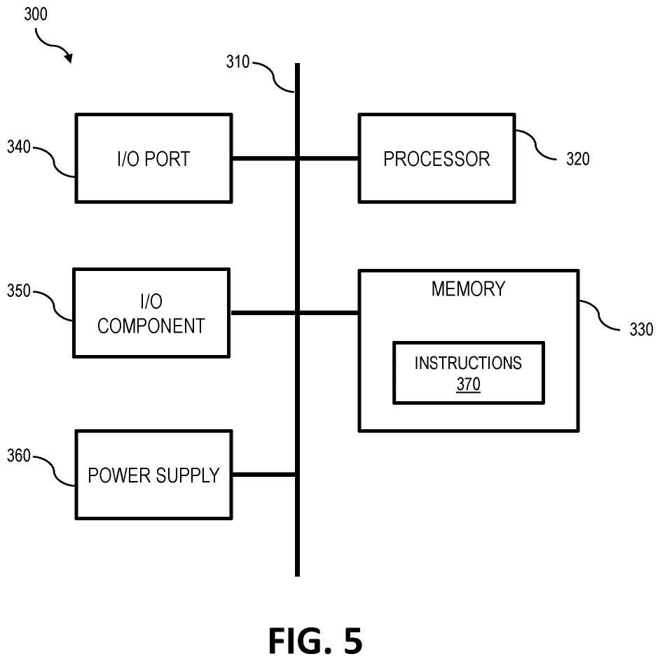

FIG. 5 is a block diagram depicting an illustrative computing device 300, in accordance with instances of the disclosure. The computing device 300 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 5 and described below. For example, the monitor 102, the mobile device 104, the server 106, and the remote computer 116 can each include their own set of components shown in FIG. 5 and described below.

In instances, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in instances, the computing device 300 may include a number of processors 320, a number of memory components 330, a number of I/O ports 340, a number of I/O components 350, and/or a number of power supplies 360. Additionally, any number of these components, or combinations thereof, may be distributed and/or duplicated across a number of computing devices.

In instances, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 330 stores computer-executable instructions 370 for causing the processor 320 to implement aspects of instances of components discussed herein and/or to perform aspects of instances of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 370 may be configured to be executed by the processor 320 and, upon execution, to cause the processor 320 to perform certain processes. In certain instances, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a server comprising one or more processors and computer-readable media having computer-executable instructions configured to be executed to cause the server to:
generate, by one or more machine learning models, a first set of classifications for a first strip of electrocardiogram (ECG) data;
generate, by the one or more machine learning models, a second set of classifications for a second strip of ECG data;
generate, by the one or more machine learning models, one or more new classifications in response to inputting only a portion of the first strip of ECG data along with only a portion of the second strip of ECG data into the machine learning model; and
update the first set of classification and/or the second set of classifications with the one or more new classifications.

2. The system of claim 1, wherein the portion of the first strip of ECG data and the portion of the second strip of ECG data comprise a beat that starts in the first strip and ends in the second strip.

3. The system of claim 2, wherein the one or more new classifications includes a classification of the beat that starts in the first strip and ends in the second strip.

4. The system of claim 1, wherein the first set of classifications include a first set of beat classifications associated with beats within the first strip, wherein the second set of classifications include a second set of beat classifications associated with beats within the second strip, wherein the one or more new classifications are beat classifications for one or more beats that overlap the first strip and the second strip.

5. The system of claim 1, wherein the computer-executable instructions are configured to be executed by the one or more processors to cause the server to:
generate, by the one or more machine learning models, a first rhythm classification associated with the first strip of ECG data.

6. The system of claim 5, wherein the computer-executable instructions are configured to be executed by the one or more processors to cause the server to:
update the first rhythm classification with a new rhythm classification based on the one or more new classifications.

7. The system of claim 6, wherein the updating the first rhythm classification with the new rhythm classification is further based on at least some of the first set of classifications.

8. The system of claim 1, wherein the portion of the first strip of ECG data contains 0.5-3 seconds of ECG data, wherein the portion of the second strip of ECG data contains 0.5-3 seconds of ECG data.

9. The system of claim 8, wherein the first strip of ECG data comprises 1-10 minutes of ECG data, wherein the second strip of ECG data comprises 1-10 minutes of ECG data.

10. The system of claim 1, wherein the one or more machine learning models comprise a deep convolutional neural network and/or a deep fully connected neural network.

11. The system of claim 10, wherein the update to the first set of classification and/or the second set of classifications with the one or more new classifications comprises updating only a subset of the first set of classification and/or the second set of classifications.

12. The system of claim 1, wherein the portion of the first strip of ECG data and the portion of the second strip comprise a continuous section of the ECG data.

13. A method comprising:
generating, by one or more machine learning models, a first set of classifications for a first strip of electrocardiogram (ECG) data;
generating, by the one or more machine learning models, a second set of classifications for a second strip of ECG data;
generating, by the one or more machine learning models, one or more new classifications in response to inputting only a portion of the first strip of ECG data along with only a portion of the second strip of ECG data into the machine learning model; and
updating the first set of classification and/or the second set of classifications with the one or more new classifications.

14. The method of claim 13, wherein the portion of the first strip of ECG data and the portion of the second strip of ECG data comprise a beat that starts in the first strip and ends in the second strip, wherein the one or more new classifications includes a classification of the beat that starts in the first strip and ends in the second strip.

15. The method of claim 13, further comprising:
generating, by the one or more machine learning models, a first rhythm classification associated with the first strip of ECG data; and
updating the first rhythm classification with a new rhythm classification based on the one or more new classifications.

16. The method of claim 15, wherein the updating the first rhythm classification with the new rhythm classification is further based on at least some of the first set of classifications.

17. The method of claim 13, wherein the portion of the first strip of ECG data contains 0.5-3 seconds of ECG data, wherein the portion of the second strip of ECG data contains 0.5-3 seconds of ECG data.

18. The method of claim 13, wherein the portion of the first strip of ECG data and the portion of the second strip comprise a continuous section of the ECG data.

19. The method of claim 13, wherein the one or more machine learning models comprise a deep convolutional neural network and/or a deep fully connected neural network.

20. The method of claim 13, wherein the updating the first set of classification and/or the second set of classifications with the one or more new classifications comprises updating only a subset of the first set of classification and/or the second set of classifications.
